(19) United States
(12) Patent Application Publication
(10) Pub. No.: US 2019/0091439 A1
(43) Pub. Date: Mar. 28, 2019

(54) A URINARY CATHETER COMPRISING AN INFLATABLE RETENTION MEMBER

(71) Applicant: Madeleine Ramstedt, Umeå (SE)

(72) Inventors: Maja Hedlund, Ursviken (SE); Joakim Bergbom, Umeå (SE)

(21) Appl. No.: 16/085,114

(22) PCT Filed: Mar. 16, 2017
(86) PCT No.: PCT/EP2017/056222

§ 371 Date: Sep. 14, 2018

(30) Foreign Application Priority Data
Mar. 17, 2016 (SE) 1650362-5

(51) Int. Cl. A61M 25/00; A61M 25/10

(52) U.S. Cl. CPC A61M 25/0017; A61M 2210/1089; A61M 25/1002; A61M 25/0068

(57) ABSTRACT

The present disclosure relates to a urinary catheter comprising a catheter tube having a proximal end and a distal inlet end portion comprising a rounded catheter tip. The distal inlet end portion further comprises a lateral urine inlet opening and the catheter tube comprises a drainage channel in fluid communication with the urine inlet opening. The catheter furthermore comprises an inflatable retention member at the distal inlet end portion, wherein the inflatable retention member is in fluid communication with an inflation channel. The distal inlet end portion is provided with a curved internal cavity. The curved internal cavity furthermore extends in a longitudinal direction along the distal inlet end portion, over the rounded catheter tip and extends further along the opposing side of the distal inlet end portion in a longitudinal direction. The inflatable retention member is formed by said curved internal cavity.

### TECHNICAL FIELD

The present disclosure pertains to a urinary catheter of the indwelling type. More specifically, the present disclosure pertains to a urinary catheter of the indwelling type provided with an inflatable retention member.

### BACKGROUND OF THE INVENTION

Catheters have been used for a very long time as a device for treating diseases or performing surgical procedures. Catheters are usually thin tubes, that may be used to inject or drain fluids and/or liquids from the body, such as for draining blood or for injecting medicine into the body.

A common use of a catheter is in the form of a urinary catheter, where a thin hollow tube is inserted into the urinary bladder via the urethra in order to void the bladder of urine that has been collected in the urinary bladder. There are two general usages of urinary catheters, where the first use is intermittent catheterization and the second use is permanent catheterization. Intermittent catheterization is where an intermittent catheter is inserted by the user into the urethra to drain the bladder, and after use the catheter is removed from the body. An intermittent catheter is a relatively stiff catheter having a hydrophilic or gel coating for lubrication and allows the users to perform the insertion on their own. Permanent catheterization is where a relatively soft and flexible catheter (indwelling catheter) is inserted into the body, and is anchored inside the body using e.g. a balloon at the tip of the catheter, so that the catheter is maintained in place for a significant period, i.e. days or weeks at a time, in order to have permanent drainage of the bladder and to reduce the need of multiple insertions of catheters for healthcare personnel.

Indwelling catheters are often made of a tubular material, that has one or more lumens where one of the lumens is for providing a fluid communication from the distal end to the proximal end in order to drain fluids, and a second lumen is often provided in order to allow a balloon or a fixation device which is attached in the vicinity of the tip (distal end) to be activated in order to ensure that the catheter is fixed in place inside the urinary bladder. The tubular material of an indwelling catheter is often made of a very flexible and soft material, so that the catheter does not restrict, prevent or oppose any movement of the urinary tract, and is designed to follow the movement of the anatomical structure surrounding the catheter, such as the urethra, urinary bladder, or the external structures of the urinary system, such as the genitalia. Indwelling urinary catheters may comprise parts extending externally from the user body, such as urinary catheter wherein a proximal end of the catheter tube is adapted to at least partly extend outside the user body during use, and may also be completely internally housed within the user body during use, such urinary catheters are also sometimes referred to as urinary stents or prostatic stents, and may be inserted into the urethra to prevent obstruction of the urethra and thereby drain urine from the bladder

Urinary catheters of the indwelling type generally are provided with an inflatable retention member disposed at a distal inlet end portion of the catheter to anchor the catheter in the bladder, the catheter including at least one drainage channel to drain urine from the bladder and at least one inflation channel to inflate the retention member (e.g. with sterile water). The proximal end of a urinary catheter of the indwelling type furthermore generally includes at least two ports in fluid communication with the two channels, a first port connected to the drainage channel and having an interface for fittings for drainage and taking of samples and a second port which is connected to the inflation channel via a valve to ensure that the inflation fluid remains within the channel and in the retention member once filled. The tip of the distal inlet end portion of a conventional urinary catheter of the indwelling type extends beyond the location of the retention member into the bladder and includes one or more apertures/urine inlet openings to drain urine from the bladder.

A consequence of the inflatable retention member being positioned below the urine inlet opening is a risk for incomplete emptying of the bladder. Moreover, the underpressure created during emptying of the bladder may cause damage to the bladder wall, as the wall may be sucked into the lateral urine inlet opening and additionally as the tip at the distal inlet end is relatively hard and may cause abrasion of the bladder wall.

Additionally, during insertion of the catheter tube into the urethra an important feature of the urinary catheter is that the catheter may slide easily through the urethra without exposing the urethral wall to any risk of damage, hence there is exists a need to improve the smoothness of urinary catheters.

### SUMMARY OF THE INVENTION

One object of the present disclosure is to provide a urinary catheter of the indwelling type with an improved and facilitated insertion and removal through the urethra and additionally with an improved protection and drainage of the urinary bladder.

This and other objects of the present disclosure may be achieved by a urinary catheter according to claim 1. Further embodiments are set out in the dependent claims, in the following description and in the drawings.

As such, the present disclosure relates to a urinary catheter comprising a catheter tube having a proximal end and a distal inlet end portion comprising a rounded catheter tip. The distal inlet end portion further comprises a lateral urine inlet opening and the catheter tube comprises a drainage channel in fluid communication with the urine inlet opening. The catheter furthermore comprises an inflatable retention member at the distal inlet end portion, wherein the inflatable retention member is in fluid communication with an inflation channel, and wherein the inflatable retention member has a non-inflated state and an inflated state. The distal inlet end portion is provided with a curved internal cavity having a length l₁. The curved internal cavity furthermore extends in a longitudinal direction along the distal inlet end portion, over the rounded catheter tip and extends further along the opposing side of the distal inlet end portion in a longitudinal direction. The inflatable retention member is formed by said curved internal cavity.

The fact that the distal inlet end portion is provided with a curved internal cavity forming the inflatable retention member provides the distal inlet end portion with a more even and smooth outer surface, as the inflatable retention member is an integrated part of the distal inlet end portion. In the non-inflated state, the inflatable retention member does not form a protruding part or constitute additional layers of material adding to the thickness of the distal inlet end portion of the catheter. Instead, the integrated inflatable retention member with the internal cavity having an outer wall formed from an expandable material provides the distal inlet end portion with a slim shape making the catheter easy to insert.

A further advantage which is linked to the inflatable retention member being formed by a curved internal cavity which extends in a longitudinal direction along the distal inlet end portion, over the rounded catheter tip and extends further along the opposing side of the distal inlet end portion in a longitudinal direction is that once the inflatable retention member is inflated the catheter tip of the distal inlet end portion is surrounded and protected by the inflated retention member.

Additionally, the fact that the inflatable retention member is not arranged at a level beneath the urine inlet opening has the effect of allowing the urine inlet opening of the inserted catheter to be located in close proximity to the urine bladder neck, which improves emptying of the bladder and prevents post void residual urine from accumulating near the bladder neck. This furthermore allows for the provision of a more elongated urine inlet opening, providing enhanced urine drainage capacity. The urine inlet opening may have an extension in the longitudinal direction of between 5 and 15 millimetres.

By “urinary catheter” herein is meant a device including a thin tube which is adapted to be inserted into the urethra and the urinary bladder to void the bladder of urine that has been collected in the urinary bladder. The term urinary catheter includes both urinary catheters partly extending externally from the user body, such as urinary catheter wherein the proximal end of the catheter tube is adapted to at least partly extend outside the user body during use, and also urinary catheters which are adapted to be completely internally housed within the user body during use, such urinary catheters are also sometimes referred to as urinary stents or prostatic stents, and may be inserted into the urethra to prevent obstruction of the urethra and thereby drain urine from the bladder.

By “lateral” urine inlet opening is meant that the urine inlet opening is positioned at the side wall of the catheter tube and thus not at the rounded catheter tip. The catheter may be provided with a urine inlet opening extending through the catheter, i.e. that the urine inlet openings provided at diametrically opposing sides, such that the urine additionally may flow through the catheter tube from one side to the other.

By “rounded” catheter tip is meant that the catheter tip has a round or curved shape, i.e. that it does not comprise any sharp corners or edges.

By a “curved” internal cavity is that the internal cavity is wrapped or bent over the distal inlet end portion with one part of the internal cavity located on either side of the catheter tip and one part of the internal cavity extending over the rounded catheter tip. The outer expandable wall of the internal cavity, contributes to defining the shape of the distal inlet end portion of the catheter. As used herein, a curved internal cavity does only have to have a curvature within the portion of the internal cavity extending over or along the rounded catheter tip.

Optionally, the curved internal cavity extends along diametrically opposed sides of the distal inlet end portion, such that the curved internal cavity has an inverted U-shape as viewed in a direction towards the rounded tip of the catheter. It may be preferred that the portions of the curved internal cavity which extend in the length direction of the catheter tube on either side of the rounded tip of the catheter have equal lengths, such that the curved internal cavity is symmetrically disposed about the distal inlet end portion of the catheter.

The fact that the curved internal cavity extends along diametrically opposed sides of the distal inlet end portion provides for equal expansion of the inflatable retention member on both sides of the urine inlet opening, which improves the positioning of the distal inlet end portion within the urinary bladder as it prevents sideways tilting of the urinary catheter and keeps the urine inlet opening in an upright position away from the bladder walls.

Optionally, the curved internal cavity, forming the inflatable retention member, extends at each side of the distal inlet end portion from positions which are further away from the rounded catheter tip than the position of said urine inlet opening. Optionally, the curved internal cavity forming the inflatable retention member extends at each side of the distal inlet end portion from positions located between 1 millimetre and 10 millimetres further away from the rounded catheter tip than the urine inlet opening such that once the retention member is inflated the urine inlet opening may be brought to a position only slightly above the bladder neck. Thereby the bladder wall at the bladder neck portion is prevented from sucked into the urine inlet opening.

Optionally, the curved internal cavity comprises an internal cavity inner wall and an internal cavity outer wall.

Optionally, the internal cavity inner wall has a cross-sectional radius of curvature, such as a radius of curvature of 2-10 millimetres, or 2-8 millimetres.

Optionally, the internal cavity outer wall has a cross-sectional radius of curvature, as measured when the inflatable retention member is in the uninflated state, such as a radius of curvature of 1.5-10 millimetres.

As the internal cavity inner wall and the internal cavity outer wall have a cross-sectional radius of curvatures, sharp edges and/or corners are prevented which improves the evenness and smoothness and also reduces unwanted bacterial growth within the inflatable retention member.

The outwardly expansion of the inflation member is mainly due to the difference in wall thickness between the inner and outer cavity walls, however this may also be obtained by using material with different stiffness, i.e. the internal cavity inner wall being stiffer or alternatively thicker than the internal cavity outer wall.

Optionally, the curved internal cavity has a length of at least 10 millimetres, or a length of from 10 millimetres to 35 millimetres.

The length of the internal cavity is measured between the positions at the surface of the catheter tube which are located furthest away from the catheter tip. Accordingly, the distance is measured from a first position at one side of the distal inlet end portion along the outer surface of the internal cavity and over the catheter tip when the inflatable retention member is provided in a non-inflated state, and to the corresponding position at the opposing side of the distal inlet end portion.

The fact that the curved internal cavity has a length of at least 10 millimetres may provide for an improved protection of the catheter tip, in the inflated state of the retention member.

The fact that the internal cavity is curved gives a curved, such as a U-shaped retention member, once inflated.

Optionally, the retention member when provided in an uninflated state forms a flush outer surface on the distal inlet end portion.

Expandability of the inflatable retention member, such as expandability of the inner cavity outer wall, is provided by a material which may be expanded or stretched, such as a material which may be elastically expanded or stretched and may be composed of a synthetic material such as biologically compatible silicone.

Optionally, the catheter tube and the inflatable retention member are made of the same material. This material may be silicone.

When the internal cavity inner wall and the internal cavity outer wall is made from the same material the outwardly expansion is due the fact that the internal cavity outer wall is much thinner than the internal cavity inner wall.

Optionally, the catheter tube and the inflatable retention member are molded as an integral unit using a synthetic material such as biologically compatible silicone. When the catheter tube and the inflatable retention member are made of the same material and/or molded as an integral unit the inflatable retention member is formed by the external walls surrounding the curved internal cavity on the side facing the user during use of the urinary catheter

### BRIEF DESCRIPTION OF THE DRAWINGS

The present invention will be further explained hereinafter by means of non-limiting examples and with reference to the appended drawings wherein:

FIG. 1 illustrates a urinary catheter according to the present disclosure;

FIG. 2 illustrates a perspective view of the distal inlet end portion;

FIG. 3 illustrates a cross-section taken along the line III-III in FIG. 2;

FIG. 4 illustrates a perspective view of the distal inlet end portion without the outer wall of the inflatable retention member;

FIG. 5 illustrates a perspective view of the distal inlet end portion with the inflatable retention member in an inflated state.

### DETAILED DESCRIPTION OF PREFERRED EMBODIMENTS

It is to be understood that the drawings are schematic and that individual components are not necessarily drawn to scale. The urinary catheters shown in the figures are provided as examples only and should not be considered limiting to the invention. Accordingly, the scope of invention is determined solely by the scope of the appended claims.

As used herein, “distal end” refers to the end of the urinary catheter furthest away from the medical operator inserting the catheter in the urethra of a patient. Thus, the distal end of the catheter is the portion which is configured to be positioned in the bladder of the patient when in use.

As used herein, “proximal end” refers to the end of the urinary catheter closest to the medical operator inserting the catheter in the urethra of a patient. Thus, the proximal portion of the catheter is the end which is configured to be positioned externally of the bladder of the patient when the catheter is in use. The proximal end of the catheter may be positioned in the urethra or outside the user when the catheter is in use.

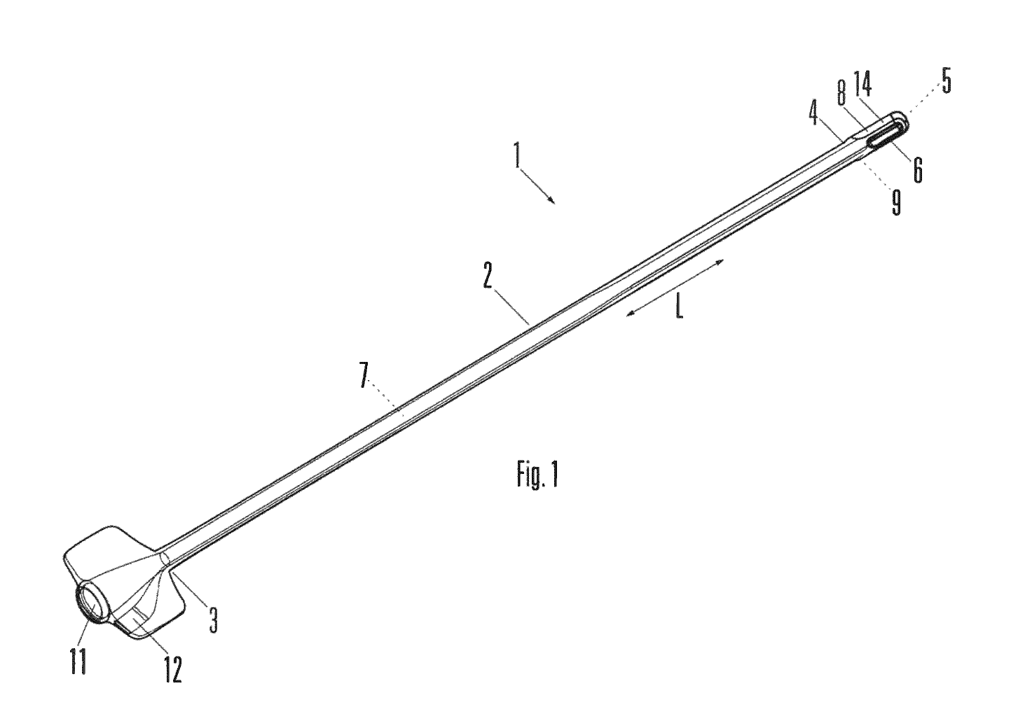

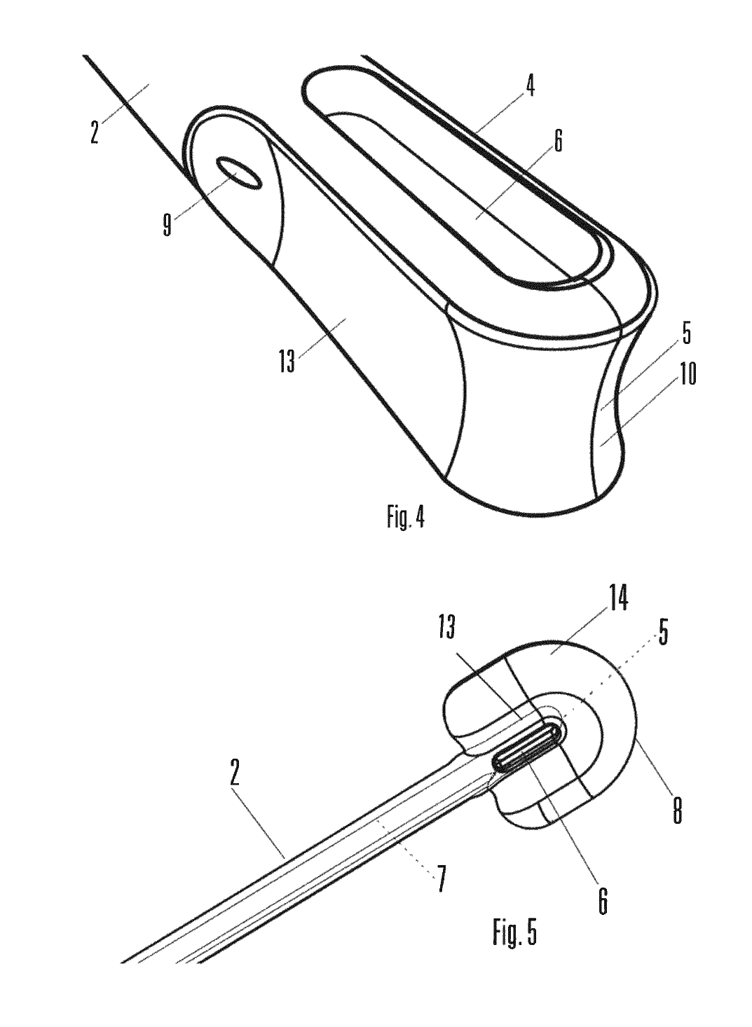

FIG. 1 illustrates a urinary catheter 1 of the indwelling type according to the present disclosure. The urinary catheter 1 comprises a catheter tube 2 having a proximal end 3 and a distal inlet end portion 4. The distal inlet end portion 4 comprises a rounded catheter tip 5, as shown in FIG. 4. The distal inlet end portion 4 furthermore comprises a lateral urine inlet opening 6 and the catheter tube 2 comprises a drainage channel 7 in fluid communication with the urine inlet opening 6. The drainage channel 7 provides fluid communication between the distal inlet end portion 4 and the proximal end 3. The urinary catheter 1 furthermore comprises an inflatable retention member 8 at the distal inlet end portion 4, wherein the inflatable retention member 8 is in fluid communication with an inflation channel 9. The inflation channel 9 provides fluid communication between the proximal end and the distal inlet end portion. The retention member 8 is shown in a non-inflated state. The proximal end 3 includes a drainage port 11 and an inflation port 12. The drainage port 11 may be provided with means for connecting the drainage port 11 to a urine collection bag or to a sampling device. The inflation port 12 may be provided with a valve to ensure that the inflation fluid remains within the channel and inflatable retention member 8 once filled. The inflatable retention 8 member may for example be inflated by sterile water.

The distal inlet end portion 4 is provided with a curved internal cavity 10. The curved internal cavity 10 furthermore extends in a longitudinal direction L along the distal inlet end portion 4, over or along the rounded catheter tip 5 and extends further along the opposing side of the distal inlet end portion 4 in a longitudinal direction L, as disclosed herein. The inflatable retention member 8 is formed by the curved internal cavity 10 comprising a rigid inner wall and an expandable cavity outer wall 14.

In an aspect of the invention, the urinary catheter may also be adapted to be a completely internal urinary catheter, such as a urinary stent, thus not comprising any parts extending outside the body of the user after being inserted into the urethra and the urinary bladder. Such urinary stent may comprise a shorter catheter tube having a drainage opening and an inflation port provided in the proximal end of the catheter tube for allowing inflation of the inflatable retention member with inflation fluid via the inflation channel. Such an inflation port may also be provided with a valve to ensure that the inflation fluid remains within the channel and the inflatable retention member once filled. The inflation port may furthermore be provided with coupling means, allowing connection to the inflation port for inflation of the retention member. Optionally, such urinary stent may be provided with a protruding retention portion, adapted to be positioned below the sphincter muscle for retention of the urinary catheter in a correct position during use.

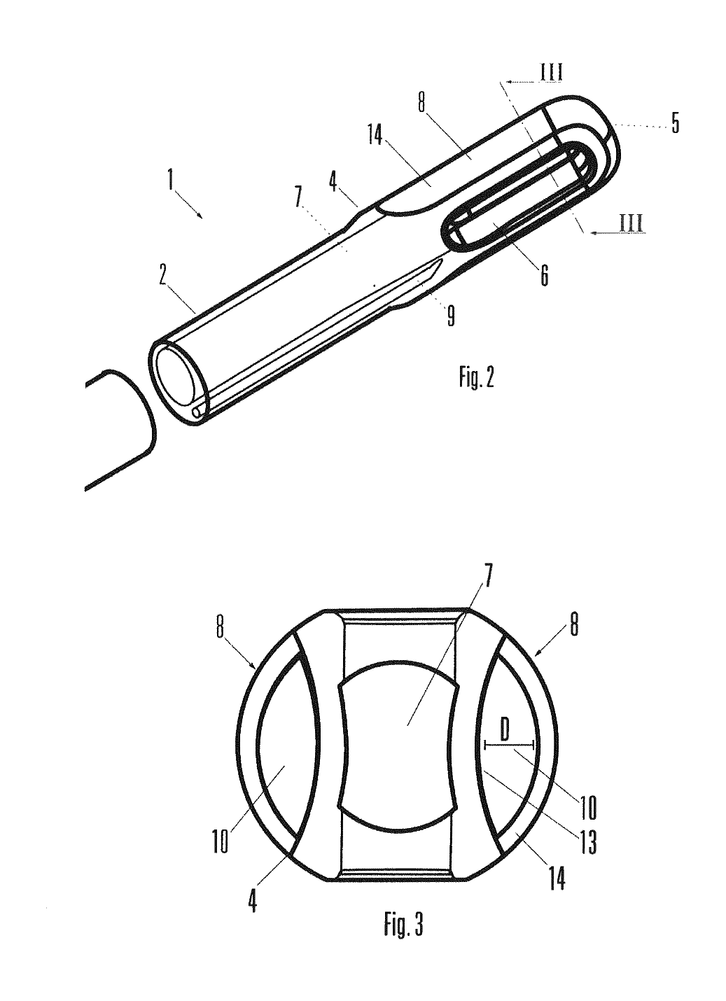

FIG. 2 illustrates a distal inlet end portion 4 of a urinary catheter 1 according to the present disclosure. The distal inlet end portion 4 is provided with a curved internal cavity 10 having a length l₁. The curved internal cavity 10 is shown to be arranged in a symmetrical manner at the distal inlet end portion 4 and extends in a longitudinal direction L along the distal inlet end portion 4, over or along the rounded catheter tip 5 and further extends along the opposing side of the distal inlet end portion 4 in a longitudinal direction L. The inflatable retention member 8 is formed by said curved internal cavity 10.

The curved internal cavity 8 and thus the inflatable retention member 8 shown in FIG. 2 extends along diametrically opposed sides of the distal inlet end portion 4 and has a U-shape. The curved internal cavity 10 at each side of the distal inlet end portion 4 extends from positions which are slightly further away from the rounded catheter tip 5 than the position of said urine inlet opening 6. It is to be understood that the end points of the curved internal cavity 10 may be located in level with the lower end of the urine inlet opening. The distance between the end points of the curved internal cavity 10 and the urine inlet opening 6 may be in the order of at most 2 millimetres, such as at most 1 millimeter.

FIG. 3 illustrates a cross-sectional view taken along the line III-III in FIG. 2 and shows the inflatable retention member 8 in a non-inflated state. The inflatable retention member 8 is partly formed by said curved internal cavity 10 and comprises here an expandable internal cavity outer wall 14 and a rigid internal cavity inner wall 13. Once the curved internal cavity 8 is filled with an inflation medium, such as sterile water, the internal cavity outer wall 14 is expanded and bulges outwardly, away from the internal cavity inner wall 13. The distance D between the internal cavity outer wall 14 and the internal cavity inner wall 13 increases by at least 3 millimetres, such as at least 10 millimetres, when the inflatable retention member 8 has been filled completely with an inflation medium. Typically, the inflatable retention member 8 may be filled with 10-20 ml sterile water and has a distance D, when inflated, of at least 4 millimetres, or from 4 millimetres to 20 millimetres, or from 5 millimetres to 15 millimetres. The distance D when the inflatable retention member 8 is in a non-inflated state may be from 0.4 to 3 millimetres, such as from 0.1 to 1 millimetre. The distance D is measured where the distance between the internal cavity outer wall 14 and the internal cavity inner wall 13 is largest.

The catheter tube and the inflatable retention member may be molded as an integral unit using a synthetic material such as biologically compatible silicone and the thickness of the internal cavity outer wall may typically be between 0.3 and 1 millimeter, or between 0.3 and 0.7 millimeter. This provides for a urinary catheter which is convenient to manufacture and where the inflatable retention member forms an integral part of the urine distal inlet opening and allows a satisfactory inflation of the inflatable retention member. Alternatively, an expandable outer wall member may be formed separately and attached to the distal inlet portion 4 of the urinary catheter 1 e.g. by any suitable attachment method such as by welding, fusing or by adhesive.

FIG. 4 illustrates a distal inlet portion 4 of a urinary catheter 1 according to the present disclosure in a view of the internal cavity inner wall 13 without the internal cavity outer wall 14 showing. As may be seen in FIG. 4 the curved internal cavity 8 and the internal cavity inner wall 13 have a cross-sectional radius of curvature. The cross-sectional radius of curvature is optionally between 2 and 10 millimetres. An inlet of the inflation channel 9 is provided in the curved internal cavity 10 at the internal cavity inner wall 13 to allow inflation of the inflatable retention member 8.

FIG. 5 is an illustration of the inflatable retention member 8 in an inflated state, when the curved internal cavity 8 is filled with an inflation medium, such as sterile water, the internal cavity outer wall 14 is expanded and bulges outwardly, away from the internal cavity inner wall 13. As illustrated, once the inflatable retention member 8 is inflated the rounded catheter tip 5 of the distal inlet end portion 4 is surrounded and protected by the inflated retention member 8.

1. A urinary catheter comprising a catheter tube having a proximal end and a distal inlet end portion comprising a rounded catheter tip, said distal inlet end portion further comprising a lateral urine inlet opening and said catheter tube comprising a drainage channel in fluid communication with said urine inlet opening, said urinary catheter furthermore comprising an inflatable retention member at said distal inlet end portion, said inflatable retention member being in fluid communication with an inflation channel and said inflatable retention member-having a non-inflated state and an inflated state, characterized in that said distal inlet end portion is provided with a curved internal cavity having a length l₁, said curved internal cavity extending in a longitudinal direction along said distal inlet end portion, over said rounded catheter tip and further extending along an opposing side of said distal inlet end portion in a longitudinal direction, wherein said inflatable retention member is formed by said curved internal cavity.

2. The urinary catheter according to claim 1, characterized in that said curved internal cavity extends along diametrically opposed sides of the distal inlet end portion such that said curved internal cavity is symmetrically disposed about said distal inlet end portion.

3. The urinary catheter according to claim 1, characterized in that said curved internal cavity forming said inflatable retention element extend at each side of said distal inlet end portion from a position which is further away from said rounded catheter tip than the position of said urine inlet opening.

4. The urinary catheter according to claim 1, characterized in that said curved internal cavity comprises an internal cavity inner wall and an internal cavity outer wall.

5. The urinary catheter according to claim 4, characterized in that said internal cavity inner wall has a cross-sectional radius of curvature of 2-10 millimetres.

6. The urinary catheter according to claim 4, characterized in that said internal cavity outer wall has a cross-sectional radius of curvature of 1.5-10 millimetres, measured when said inflatable retention member is in an uninflated state.

7. The urinary catheter according to claim 1, characterized in that said length l₁ of said curved internal cavity is at least 10 millimetres.

8. The urinary catheter according to claim 1, characterized in that said inflatable retention member, when being in a non-inflated state, forms a flush outer surface on the distal inlet end portion.

9. The urinary catheter according to claim 1, characterized in that said distal inlet end portion and said inflatable retention member are made of the same material.

10. The urinary catheter according to claim 1, characterized in that said catheter tube and said inflatable retention member are made of silicone.

11. The urinary catheter according to claim 1, characterized in that said catheter tube and said inflatable retention member are molded as an integral unit.
